(19) United States
(12) Patent Application Publication
(10) Pub. No.: US 2026/0066101 A1
(43) Pub. Date: Mar. 5, 2026

(54) Method for Assisting a User in Implementing an Examination Workflow of a Magnetic Resonance Examination

(71) Applicant: Siemens Healthineers AG, Forchheim (DE)

(72) Inventors: David Grodzki, Erlangen (DE); Tobias Würfl, Erlangen (DE); Mario Zeller, Erlangen (DE); Rainer Schneider, Höchstadt (DE); Thomas Möck, Nürnberg (DE); Armin Stranjak, Uttenreuth (DE)

(73) Assignee: Siemens Healthineers AG, Forchheim (DE)

(21) Appl. No.: 19/312,386

(22) Filed: Aug. 28, 2025

(30) Foreign Application Priority Data
Aug. 29, 2024 (DE) 10 2024 208 222.8

(51) Int. Cl. G16H 40/20; G16H 10/60

(52) U.S. Cl. CPC G16H 40/20; G16H 10/60

(57) ABSTRACT

The disclosure relates to a method for assisting a user in implementing a workflow of a magnetic resonance examination on a patient. The method may include receiving a query by the user, wherein the input is made in text form or as voice input; determining output information corresponding to the query by means of a large language model (LLM), and providing the output information; and outputting the output information in text form or as voice output.

### CROSS REFERENCE TO RELATED APPLICATIONS

This patent application claims priority to German Patent Application No. 102024208222.8, filed Aug. 29, 2024, which is incorporated herein by reference in its entirety.

### BACKGROUND

The present disclosure relates to a method for assisting a user in implementing a workflow of a magnetic resonance examination on a patient. The present disclosure also relates to a computing unit and to a system having a computing unit and a magnetic resonance apparatus, wherein the computing unit may be configured to perform the method for assisting a user in implementing a workflow of a magnetic resonance examination on a patient. In addition, the present disclosure also includes a corresponding computer program product and a data storage medium containing the computer program product.

A magnetic resonance examination may often be carried out on a patient in order to clarify a specific medical and/or diagnostic issue. There may be numerous options for adapting the magnetic resonance examination to the medical and/or diagnostic issue. This may require, however, a large amount of expert knowledge on the part of the medical operator supervising the magnetic resonance examination. Even small changes in the settings can lead to errors in the setting of sequence parameters. For example, a change to parameter settings can transform a sequence from proton density contrast to a T2-weighted sequence, although this may not be reflected by the name of the sequence and therefore may not be obvious to an inexperienced user. Another example is that artifacts can be caused by setting too high a parallel acquisition technique (PAT) factor.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 shows a method for assisting a user in implementing a workflow of a magnetic resonance examination on a patient according to the disclosure.

FIG. 2 shows a computing unit according to the disclosure.

FIG. 3 shows a system having a magnetic resonance apparatus and a computing unit, according to the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, where a connection can be wireless or wired. Functional units can be implemented as hardware, software or a combination of hardware and software.

An object of the present disclosure is to assist a user in implementing and/or preparing a magnetic resonance apparatus.

The disclosure provides a computer-implemented method for assisting a user in implementing a workflow of a magnetic resonance examination on a patient. The method may include: receiving a query by the user, wherein the query may be in text form or as a voice input; determining output information corresponding to the query by means of a large language model (LLM), and providing the output information; and output of the output information in text form or as voice output.

The user may be a medical operator supervising the magnetic resonance examination, for instance a doctor or a radiographer. The user, such as the medical operator, may not just supervise implementation of the magnetic resonance examination but also prepare the magnetic resonance examination. The preparation of the magnetic resonance examination may also include selecting a measurement program and specifying required settings in parameters, such as sequence parameters, to suit the clinical issue. Preparation of the magnetic resonance examination, such as selecting a measurement program and selecting the required settings, can also be performed well before the actual magnetic resonance examination.

The selection of the measurement program may be made by means of a selection module of a computing unit and/or of a controller. The computing unit and/or controller may be included in the magnetic resonance apparatus or be connected to the magnetic resonance apparatus via an interface. In addition, the computing unit and/or controller may also have a dedicated user interface for interaction with the user, such as the medical operator, or else be connected via an interface unit to a user interface of the magnetic resonance apparatus or to a user interface of further units, for example of a mobile terminal. The selection module may include selection software which, when executed by a processor (or processing circuitry) of the computing unit and/or controller, provides a user with a user interface for selecting a measurement program.

The method according to the disclosure can be performed both in the preparation of the magnetic resonance examination and during implementation of the magnetic resonance examination. A user, such as the medical operator, can access the method according to the disclosure for assistance at any time during the preparation of the magnetic resonance examination and/or during the implementation of the magnetic resonance examination.

The workflow of a magnetic resonance examination may comprise both a measurement workflow, which includes implementing the magnetic resonance examination, and a preparation workflow, which includes preparing the magnetic resonance examination. The preparation workflow that includes the preparing of the magnetic resonance examination may comprise both preparing the patient and registering the patient, and selecting a measurement program. The preparation of the patient can comprise positioning the patient on a patient table of the magnetic resonance apparatus or placing or positioning local radiofrequency coils on the patient, such as on the region to be examined of the patient, or attaching add-on units such as an infusion unit, an ECG unit, etc., for example. The registering of the patient can comprise input of patient-relevant data, which can be relevant to the selection of a measurement program and/or of individual parameter settings. The patient data can comprise a size of the patient, an age of the patient, a progression of a disease in a patient, a region to be examined of the patient, and/or further patient data that the professional deems useful. The selection of the measurement program may be made based on the clinical and/or diagnostic issue to be clarified by the magnetic resonance examination. In addition, the selection of the measurement program can also depend on a region to be examined of the patient. The selected measurement program may comprise a succession of different measurement sequences, which are implemented one after the other in a defined order. For this purpose, the preparation can also comprise selecting or setting individual parameters for different measurement sequences.

The measurement workflow of the magnetic resonance examination may comprise the implementation of the measurement program, such as the implementation of the different measurement sequences one after the other. This may also require interactions by the user, such as by the medical operator. For example, it can be provided that a contrast agent infusion for a contrast agent measurement has to be started by a medical operator at a defined point in time in the measurement process. It can also be the case that the medical operator has to assess an image quality of captured image data as to whether the image quality is adequate for a diagnosis. If the image quality is not adequate, the medical operator must decide whether the current measurement step is aborted and restarted, or whether an alternative measurement strategy is used for the rest of the magnetic resonance examination, etc.

For all these actions during the preparation workflow and during the measurement workflow, the medical operator can input a query for assistance or if questions or problems arise.

The determination of the output information corresponding to the query may be performed by means of a computing unit, which has a determination module containing a large language model (LLM). The computing unit may include the determination module containing the LLM can be the same computing unit that includes the selection module. In addition, it is also conceivable that the computing unit including the determination module containing the LLM may be separate from the computing unit containing the selection module. Furthermore, the computing unit containing the determination module may also be included in the magnetic resonance apparatus. Alternatively, the computing unit containing the determination module may also be separate from the magnetic resonance apparatus and be connected to the magnetic resonance apparatus via an interface. For example, a cloud computing system may also include the computing unit.

The LLM may be configured and/or trained to analyze an input by the user, such as the query by the user, and to create context-related responses. For example, the LLM may analyze the query for key words, and create relevant or corresponding output information based on the key words. Alternatively, or additionally, the LLM may analyze the query also in the context of a current workflow status. For example, for this purpose, the determination module may retrieve a current workflow status and provide it to the LLM for determining the output information. The LLM may also take into account in the analysis of the query and in determining the output information, earlier queries on this topic and output information determined therefore, and/or an assessment made by the medical operator of the output information from previous magnetic resonance examinations. Such queries and their corresponding output information may be stored in a memory unit of the determination module. The determination module provides the determined output information for output. It provides the output information to an output unit of the user interface.

The LLM may represent a computer linguistics probabilistic model, which has learned statistical word-order and sentence-order relationships from a large number of text documents through a computationally intensive training process.

The input of the query and the output of the output information may be made via a user interface, which has an input unit and an output unit for this purpose. The input unit may include a keyboard and/or a touch-display for input of the query in text form. In addition, the input unit may also include a microphone for input of the query as a voice message. The output unit may include a monitor and/or a display for output of the output information in text form. In addition, the output unit may also include loudspeakers for output of the output information as a voice message. Information in text form shall be understood to mean here that the information exists in written form and/or is readable.

The query may also be input here in natural language. The query may be made without precise knowledge of specialized terminology for magnetic resonance examinations. For example, it may be possible to ask what is causing the duplication of organ outlines without using the key terms “aliasing” or “parallel imaging”. This may result in a far higher success rate in solving usage problems. Assistance can be given easily in this way to an inexperienced medical operator who has not yet mastered the precise specialized terminology.

For example, for input of the query, a communication window may be shown on a display continuously during the entire workflow of the magnetic resonance examination. Such a communication window may be shown via a user interface or shown as a pop-up window. Such a communication window may include an input region for input of the query by the user. For example, the query may be input in text form and/or a voice input can be activated via said communication window. In addition, the output information may also be output via the communication window. In this case, it may also be indicated to the user that the output information exists as a voice message, so that the user can retrieve the output information when required.

The disclosure may provide advantageous assistance to a user, such as to a medical operator, for a magnetic resonance examination. For example, by targeted querying, the medical operator may be informed of the effect of individual configuration options, and which constraints may be observed here.

In an advantageous development of the method according to the disclosure, at least one piece of additional information is provided, which the LLM takes into account in the determining of the output information, wherein this at least one piece of additional information includes: information on the current magnetic resonance examination; information on at least one previous magnetic resonance examination; a hardware attribute of the magnetic resonance apparatus; a software attribute of the magnetic resonance apparatus; and/or information on the patient.

The information on the current magnetic resonance examination may include current parameter settings of sequence parameters. In addition, the information may also include a current workflow status. For example, this may provide a context in which the query by the medical operator originates and hence also reduce a selection of output information.

The information on a previous magnetic resonance examination may include information about usage during one or more previous magnetic resonance examinations in an identical or similar situation to the query context. For example, for queries about parameter settings, settings from previous magnetic resonance examinations may be taken into account. Information from previous magnetic resonance examinations using an identical or similar measurement program may be taken into account here.

The hardware attribute of the magnetic resonance apparatus may include, for example, information relating to the radiofrequency coils that are used, or intended to be used, for the magnetic resonance examination. For example, the information may relate to whether the correct radiofrequency coils are used for the selected measurement program and whether they are also positioned and/or connected correctly. In addition, the hardware attribute of the magnetic resonance apparatus may also include information on a magnetic field strength and/or information relating to a maximum available gradient field strength, etc.

The software attribute of the magnetic resonance apparatus may include information relating to existing software licenses. For example, response information to a query about setting sequence parameters may be dependent on available measurement options, which can be associated with a software license.

The information on the patient may include a weight of the patient and/or an age of the patient and/or a body size of the patient and/or a progression of a disease in the patient and/or implant information on the patient and/or further information and/or attributes of the patient that the professional deems useful.

The at least one piece of additional information may be provided to the LLM automatically by the computing unit, such as by the determination module, in order to generate output information based on the current query combined with the at least one piece of additional information. The additional information may include existing data and/or data stored and/or held for implementing the magnetic resonance examination, which the LLM can access in order to analyze the query and determine the output information.

This may increase the likelihood of providing response information that can contribute to solving a current issue and/or a current problem and/or may bring about an improvement to a current situation.

In an advantageous development of the method according to the disclosure, the output information includes a connecting element, for instance a link, to a defined passage of text in stored documentation, wherein when the user activates the connecting element by means of a user interface, a connection is established to the stored documentation, and the defined passage of text is output to the user. For example, the documentation includes user documentation in which are documented individual steps for setting and/or implementing individual measurement steps of a measurement program. The activation may be performed by clicking with a computer mouse and/or a finger of the medical operator on a display, such as a touch-display, and/or by voice input. A linkage to a defined passage of text in documentation may provide additional verification of and/or support for the response information provided to the user, such as to the medical operator, and hence engender and/or produce a sense of security for the medical operator in a critical situation.

In an advantageous development of the method according to the disclosure, the output information includes at least one additional suggestion for current settings of the current magnetic resonance examination. For example, for a query relating to a setting of sequence parameters, it can be indicated to the medical operator what would be the ideal or optimum setting of the sequence parameters, and what, in contrast, the current selection of the setting of the sequence parameters includes. It is thus also possible to show the medical operator associations between individual sequence parameters if he were to have a particular setting of a sequence parameter, but this is blocked by a value of a further sequence parameter.

In an advantageous development of the method according to the disclosure, in a further method step, a further input by the user can be input in response to the output information. The further input can include feedback information from the user, which feedback information is fed to the LLM for assessment of the determined and provided output information. It is thereby advantageously possible to provide the LLM with feedback information from the user relating to the output information created by the LLM. The feedback information can include, for example, information on how well the output information has helped the user with their current specific problem and contributed to solving the problem. The LLM for determining the output information can thus be continuously refined and retrained in order to determine the best possible output information for a query by a user. For example, in the case of queries for which a selection can be made from a plurality of possible output information, the LLM can rank which of the output information best contributes to solving the current problem or to answering the query. This also allows the LLM to react dynamically to changes and to discard existing output information, for instance proposed solutions, or to add new output information.

For example, output information containing guidance that has not contributed to solving the specific problem of the user and/or has made only a minor contribution to improving the current situation, is assessed negatively, and in the event of a new query with an identical problem is no longer provided as the first solution approach or as the first output information. On the other hand, output information containing guidance that has proved helpful and contributed to solving the current problem is assessed positively, and in the event of a new query with an identical problem is again provided as the first solution approach, such as the first output information.

In an advantageous development of the method according to the disclosure, further output information is created and output based on the further input. For example, in the case of a negative assessment of the first output information, new output information can be determined and provided. This makes it possible to provide comprehensive assistance for a user, such as a medical operator, even if the first output information containing a first proposed solution has not contributed to solving the current problem or to clarifying the query. For example, in the case of duplicated structures, if the minimum possible PAT factor has already been used, the further input can ask what other measure can bring about an improvement.

In an advantageous development of the method according to the disclosure, the further output information includes contact information relating to a human contact. If it has not yet been possible to provide a satisfactory solution to the query by the user, this allows the user to contact an expert to solve the query. In addition, it may also be the case that in addition to the providing and output of the contact data, contact is made as well to the expert.

In addition, the disclosure provides a computing unit, which is configured to assist a user in implementing a workflow of a magnetic resonance examination on a patient, wherein the computing unit includes: an interface module, which is configured for connection to a user interface, wherein the user interface has an input unit for input of a query by a user, wherein the input is made in text form or as voice input; a determination module containing an LLM, which is configured to determine output information corresponding to the query; and a provision module, which is configured to provide the output information to an output unit.

The advantages of the computing unit according to the disclosure are essentially the same as the advantages detailed above of the method according to the disclosure for assisting a user in implementing a workflow of a magnetic resonance examination on a patient. The claims for the computing unit can be improved by features that are described or claimed in connection with the method, and vice versa. In this case, structural units of the computing unit embody the functional features of the method, and vice versa.

The interface module may include an interface for connecting to a user interface. The user interface may comprise an input unit for input of a query by the user. The user can make the query here in text form or as voice output. For this purpose, the input unit has a microphone and/or a keyboard and/or a touch-display, etc. for the input of the query.

The provision module may include an interface, which interface may be configured for a data connection to a user interface. The user interface has for this purpose an output unit, which may be configured for output in text form or as voice output. The output unit may include a display and/or loudspeaker.

The provision module and the interface module can also be integral with each other if an input unit for the input of the query by a user and the output unit for the output of the provided output information are comprised by a common user interface.

The computing unit may be configured to perform the above-described method for assisting a user in implementing a workflow of a magnetic resonance examination on a patient. For example, the interface can be used to show continuously a communication window for input of the query and for output of the output information in a display of a user interface of the measurement program.

The computing unit according to the disclosure has the advantage of being able to provide advantageous assistance to a user, such as to a medical operator, for a magnetic resonance examination. For example, by targeted querying, the medical operator can be informed of the effect of individual configuration options and which constraints must be observed here.

In addition, the disclosure provides a system including a magnetic resonance apparatus and a computing unit, which computing unit is configured as described above, wherein the system is configured to perform an aforementioned method for assisting a user in implementing a workflow of a magnetic resonance examination on a patient.

The advantages of the system according to the disclosure are essentially the same as the advantages detailed above of the method according to the disclosure for assisting a user in implementing a workflow of a magnetic resonance examination on a patient. The claims for the system can be improved by features that are described or claimed in connection with the method, and vice versa. In this case, structural units of the system embody the functional features of the method, and vice versa.

In an advantageous development of the system according to the disclosure, the magnetic resonance apparatus has a user interface, which is connected to an interface module and/or a provision module of the computing unit. The user interface may include an input unit and an output unit, and is connected both to the interface module and to the provision module in order to facilitate user-input of an input of a query and the output of the output information created by the LLM.

According to a further aspect, a computer program product is provided containing program elements which cause a computing unit to perform the steps of the aforementioned method for assisting a user in implementing a workflow of a magnetic resonance examination on a patient when the program elements are loaded into a memory of the computing unit.

According to yet another aspect, a computer-readable medium is provided on which are stored program elements which can be read and executed by a computing unit in order to carry out steps of the aforementioned method for assisting a user in implementing a workflow of a magnetic resonance examination on a patient when the program elements are executed by the computing unit.

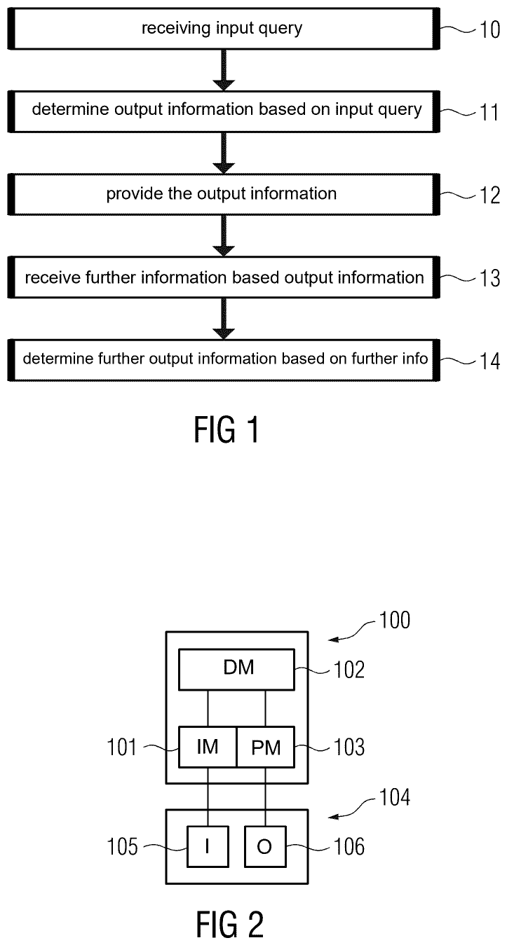

FIG. 1 shows a flow diagram of a computer-implemented method for assisting a user in implementing a workflow of a magnetic resonance examination on a patient 202.

The method may be performed by a computing unit (computer, controller) 100, which is shown in greater detail in FIG. 2. Computing unit 100 may include an interface module (interface) 101, a determination module (determininer, processor) 102, and a provision module (input/output interface) 103. Computing unit 100 may include processing circuitry that is configured to perform one or more functions and/or operations of computing unit 100. Additionally, or alternatively, one or more components of the computing unit 100 (e.g., interface module 101, a determination module 102, and/or a provision module 103) may include processing circuitry that is configured to perform one or more respective functions and/or operations of the component(s).

The interface module 101 may be configured for connection to a user interface 104, which user interface has an input unit 105 for input of a query by the user. The query by the user is made here in text form or as voice output. For this purpose, the input unit 105 has a microphone and/or a keyboard for the input of the query.

The determination module 102 may comprise an LLM. The LLM may be configured to determine output information corresponding to the query.

The provision module 103 may be configured to provide the output information determined by the determination module 102, such as the LLM, to an output unit 106 of the user interface 104. The output unit 106 may include a display and/or loudspeaker.

In an exemplary embodiment, the provision module 103 is integral with the interface module 101. The user interface 104 can be included in a magnetic resonance apparatus 201. In addition, the user interface 104 can also be separate from the magnetic resonance apparatus 201.

In a first method step 10, the user inputs a query, with the input made at the input unit 105. The input can be made in text form or as voice input. The input of the query can also be made in natural language, i.e. even without the user having precise knowledge of a specialized terminology. For example, it is possible to ask what is causing the duplication of organ outlines without using the key terms “aliasing”or “parallel imaging”.

A user can input a query during a preparation workflow of the magnetic resonance examination or during a measurement workflow of the magnetic resonance examination. The preparation workflow may comprise preparing the magnetic resonance examination, for example positioning the patient 202 and/or selecting a measurement program, etc. The measurement workflow, on the other hand, may comprise implementing the measurement program.

In a further, second method step 11, output information corresponding to the query is determined by means of the LLM of the computing unit 100. The LLM can additionally access further information for determining the output information. This further information can comprise information on the current magnetic resonance examination and/or information on at least one previous magnetic resonance examination and/or a hardware attribute of the magnetic resonance apparatus and/or a software attribute of the magnetic resonance apparatus and/or information on the patient 202.

The information on the current magnetic resonance examination can comprise current parameter settings of sequence parameters. In addition, the information can also comprise a current workflow status. For example, this can provide a context in which the query by the medical operator originates.

The information on a previous magnetic resonance examination can comprise information about a usage during one or more previous magnetic resonance examinations in an identical or similar situation to the query context. For example, for queries about parameter settings, settings from previous magnetic resonance examinations can be taken into account. In an exemplary embodiment, information from previous magnetic resonance examinations using an identical or similar measurement program may be considered here. This data from previous magnetic resonance examinations may be stored in a memory unit and/or a database, with the computing unit 100, such as the LLM, having access rights to the memory unit and/or the database. An embedding database at the system itself, such as at the computing unit 100, or even in a cloud computing system, can be used for this. Also, the information on a previous magnetic resonance examination can take into account an identical or similar query relating to a problem posed by a previous magnetic resonance examination, and feedback on the output information for a previous magnetic resonance examination can be taken into account in the determining of the output information.

The hardware attribute of the magnetic resonance apparatus 201 can comprise, for example, information relating to the radiofrequency coils that are used, or intended to be used, for the magnetic resonance examination. In addition, the hardware attribute of the magnetic resonance apparatus can also comprise information on a magnetic field strength and/or information relating to a maximum available gradient field strength, etc.

The software attribute of the magnetic resonance apparatus 201 can comprise information relating to existing software licenses. For example, response information to a query about setting sequence parameters can be dependent on available measurement options, which can be associated with a software license.

The information on the patient 202 can comprise a weight of the patient 202 and/or an age of the patient 202 and/or a body size of the patient 202 and/or a progression of a disease in the patient 202 and/or implant information on the patient 202 and/or further information and/or attributes of the patient 202 that the professional deems useful.

The at least one piece of additional information may be provided to the LLM automatically by computing unit 100, such as by the determination module 102, in order to generate output information based on the current query combined with the at least one piece of additional information. In an exemplary embodiment, the determination module 102 accesses the further information via the interface module 101.

After the output information is determined by means of the LLM, in this second method step 11 the provision module 103 provides the output information for output. It provides the output information to an output unit 106 of the user interface 104.

In a further, third method step 12, the output information is output at the output unit 106 of the user interface 104. The output at the output unit 106 is made here in text form at a display of the output unit 106 or as voice output via loudspeakers of the output unit 106.

The output information can comprise a connecting element, for instance a link, to a defined passage of text in stored documentation. When the user activates the connecting element by means of the user interface 104, a connection is established to the stored documentation, and the defined passage of text is output to the user at the output unit 106. The activation can be performed by clicking with a computer mouse and/or a finger on a display and/or by voice input. The output of the defined passage of text can likewise be made via the output unit 106, such as the display or the loudspeakers.

In addition to a connecting element, the output information can also comprise additional suggestions for current settings of the magnetic resonance examination. The current settings can comprise parameter settings that can be set by the medical operator when selecting the measurement program.

In a further, fourth method step 13, a further input by the user can be input in response to the output information. This input is made in text form or as voice input likewise by means of the input unit 105 of the user interface 104. This further input can comprise feedback information from the user, which is fed to the LLM for assessment of the created output information. In addition, the further input can comprise a further request for output information, for instance if the user has uncertainties about using and/or implementing the output information.

In a further, fifth method step 14, further output information is created by the LLM based on the further input, and is output to the user via the output unit 106 of the user interface 104. This further output information can comprise alternative guidance to the first output information and/or additional information to the first output information. In addition, the further output information can also comprise contact information relating to a human contact, such as an expert.

The fourth method step 13 and the fifth method step 14 can also be available only optionally depending on the form of the method for assisting a user in implementing a workflow of a magnetic resonance examination on a patient 202.

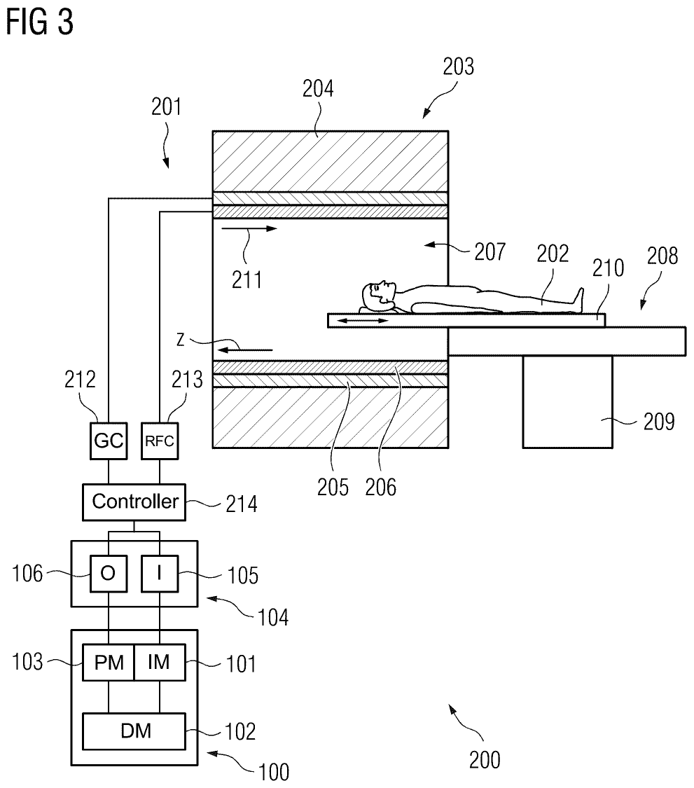

FIG. 3 shows schematically a system 200 having a magnetic resonance apparatus 201 and the computing unit 100 known from FIG. 2. The magnetic resonance apparatus 201 may comprise a magnet unit 203 having a main magnet 204, a gradient coil unit 205 and a radiofrequency antenna unit 206. The magnetic resonance apparatus 201 may also comprise a patient placement region 207 for accommodating the patient 202 for a magnetic resonance examination. In the present exemplary embodiment, the patient placement region 207 is shaped as a cylinder and is enclosed in a circumferential direction cylindrically by the magnet unit 203. In principle, however, it is always conceivable that the patient placement region 207 has a different design.

The magnetic resonance apparatus 201 has a patient positioning apparatus 208 for positioning the patient 202, such as a region to be examined of the patient 202, inside the patient placement region 207. The patient positioning apparatus 208 has a base unit 209 and a patient table 210, which can move with respect to the base unit 209. The patient table 210 may be configured to position the patient 202, such as the region to be examined of the patient 202, movably inside the patient placement region 207. The patient table 210 is mounted such that it can move in the direction of a longitudinal extent of the patient placement region 207 and/or in the z-direction.

The main magnet 204 of the magnet unit 203 may be configured to produce a powerful and constant main magnetic field 211. For example, said main magnet 204 may be in the form of a superconducting main magnet or a permanent magnet. The gradient coil unit 205 of the magnet unit 203 may be configured to produce magnetic field gradients used for spatial encoding during imaging. The gradient coil unit 205 is controlled by a gradient controller 212 of the magnetic resonance apparatus 201. The radiofrequency antenna unit 206 of the magnet unit 203 may be configured to excite a polarization, which is established in the main magnetic field 211 produced by the main magnet 204. The radiofrequency antenna unit 206 is controlled by a radiofrequency antenna controller 213 of the magnetic resonance apparatus 201 and radiates high-frequency magnetic resonance sequences into the patient placement region 207 of the magnetic resonance apparatus 201.

The magnetic resonance apparatus 201 has a system controller 214 configured to control the main magnet 204, the gradient controller 212 and/or the radiofrequency-antenna controller 213. The system controller 214 may centrally control the magnetic resonance apparatus 201, for instance implementing a predetermined imaging gradient echo sequence. In addition, the system controller 214 may comprise an analysis unit (not presented in further detail, such as processing circuitry, processor(s), or the like) configured to analyze medical image data acquired during the magnetic resonance examination. The system controller 214 may include processing circuitry that is configured to perform one or more functions and/or operations of the system controller 214. Additionally, or alternatively, one or more components of the system controller 214 may include processing circuitry that is configured to perform one or more respective functions and/or operations of the component(s).

The magnetic resonance apparatus 201 may further comprise the user interface 104, which is connected to the system controller 214. In addition, the user interface 104 of the magnetic resonance apparatus 201 is connected to the computing unit 100, such as to the interface module 101 and the provision module 103 of the computing unit 100, for input of the query and output of the output information.

The magnetic resonance apparatus 201 shown can obviously comprise further components that are typically present in magnetic resonance apparatuses 201. Furthermore, since a person skilled in the art knows how a magnetic resonance apparatus 201 works in general, a detailed description of the further components is not given.

Although the disclosure has been illustrated and described in detail using the exemplary embodiment, the disclosure is not limited by the disclosed examples, and a person skilled in the art can derive other variations therefrom without departing from the scope of protection of the disclosure.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices. ” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A computer-implemented method for assisting a user in implementing a workflow of a magnetic resonance examination on a patient, the method comprising:
receiving, by a computing unit, a query from the user, wherein the query is received in text form or as voice input;
determining, by the computing unit and using a large language model (LLM), output information corresponding to the query;
providing, by the computing unit, the output information; and
outputting, by the computing unit, the output information in text form or as voice output.

2. The method of claim 1, further comprising: receiving, by the LLM, at least one piece of additional information for use in determining the output information, wherein the at least one piece of additional information comprises:
information regarding the current magnetic resonance examination;
information regarding at least one previous magnetic resonance examination;
a hardware attribute of the magnetic resonance apparatus;
a software attribute of the magnetic resonance apparatus; and/or
information regarding the patient.

3. The method of claim 2, wherein the information regarding the patient comprises:
a weight of the patient;
a body size of the patient;
a progression of a disease in the patient;
implant information for the patient; and/or
additional attributes of the patient.

4. The method of claim 1, wherein the output information comprises a connecting element linked to a defined passage of text in stored documentation, the method further comprising:
receiving, by the computing unit, an activation of the connecting element from the user via a user interface;
establishing, by the computing unit, a connection to the stored documentation based on the activation; and
outputting, by the computing unit, the defined passage of text to the user.

5. The method of claim 1, wherein the output information comprises at least one suggestion for current settings of the current magnetic resonance examination.

6. The method of claim 1, further comprising: receiving, by the computing unit, a further input from the user in response to the output information.

7. The method of claim 6, wherein the further input comprises feedback information from the user, and wherein the method further comprises: providing, by the computing unit, the feedback information to the LLM for assessment of the determined output information.

8. The method of claim 6, further comprising creating and outputting further output information based on the further input.

9. The method of claim 8, wherein the further output information comprises contact information relating to a human contact.

10. The method of claim 1, wherein determining the output information comprises analyzing, by the LLM, the query within a context of current parameter settings of sequence parameters for a magnetic resonance examination.

11. The method of claim 1, wherein determining the output information comprises analyzing, by the LLM, radiofrequency coil configuration data to determine whether correct radiofrequency coils are positioned and connected for a selected measurement program.

12. The method of claim 1, wherein the LLM comprises a computer linguistics probabilistic model that has learned statistical word-order and sentence-order relationships from text documents through a training process, and wherein determining the output information comprises applying, by the LLM, learned statistical word-order and sentence-order relationships to analyze the query and generate contextually appropriate responses.

13. The method of claim 1, wherein determining the output information comprises retrieving, by the computing unit, a current workflow status of a magnetic resonance examination, providing the current workflow status to the LLM for contextual analysis of the query, and adjusting, by the LLM, a selection of output information based the contextual analysis of the workflow status.

14. The method of claim 1, wherein determining the output information comprises retrieving, by the computing unit, current parameter settings of sequence parameters for a magnetic resonance examination, providing the current parameter settings to the LLM for contextual analysis of the query, and determining, by the LLM and based on the current parameter settings, parameter conflicts and one or more alternative parameter settings to maintain diagnostic image quality.

15. One or more non-transitory media storing instructions that, when executed by one or more processors, cause the one or more processors to perform the method of claim 1.

16. An apparatus comprising: one or more processors; and memory storing instructions that, when executed by the one or more processors, cause the apparatus to perform the method of claim 1.

17. A computing unit configured to assist a user in implementing a workflow of a magnetic resonance examination on a patient, the computing unit comprising:
an interface module configured to connect to a user interface having an input unit configured to receive a query from the user in text form or as voice input;
a determination module including a large language model (LLM) configured to determine output information corresponding to the query; and
a provision module configured to provide the output information to an output unit for output to the user.

18. A system comprising a magnetic resonance apparatus and the computing unit of claim 17.

19. The system of claim 18, wherein the magnetic resonance apparatus comprises a user interface, which is connected to an interface module and/or a provision module of the computing unit.

20. A computer-implemented method for assisting a user in implementing a workflow of a magnetic resonance examination on a patient, the method comprising:
receiving, by a computing unit operatively connected to a magnetic resonance apparatus, a query from the user regarding magnetic resonance examination parameters, wherein the query is received in text form or as voice input;
determining, by the computing unit using a large language model (LLM) trained on magnetic resonance imaging technical data, output information corresponding to the query by analyzing the query in context of current operational parameters of the magnetic resonance apparatus;
providing, by the computing unit, the output information including specific parameter adjustments for magnetic resonance sequence settings; and
outputting, by the computing unit to a user interface of the magnetic resonance apparatus, the output information in text form or as voice output during the magnetic resonance examination workflow.
